(19) United States
(12) Patent Application Publication
(10) Pub. No.: US 2009/0105555 A1
(43) Pub. Date: Apr. 23, 2009

(54) NON-INVASIVE MONITORING OF PHYSIOLOGICAL MEASUREMENTS IN A DISTRIBUTED HEALTH CARE ENVIRONMENT

(76) Inventors: Clifford C. Dacso, Bellaire, TX (US); Nithin O. Rajan, Houston, TX (US)

Correspondence Address: BRACEWELL & GIULIANI LLP, P.O. BOX 61389, HOUSTON, TX, 77208-1389, US

(21) Appl. No.: 12/108,177

(22) Filed: Apr. 23, 2008

Related U.S. Application Data
(60) Provisional application No. 60/927,023, filed on Apr. 30, 2007.

(51) Int. Cl. A61B 5/0295; A61B 5/029

(52) U.S. Cl. 600/301; 600/526

(57) ABSTRACT

A system for determining physiological characteristics.

### 1. CROSS-REFERENCE TO RELATED APPLICATIONS

The present application claims the benefit of the filing date of U.S. provisional patent application Ser. No. 60/927,023, filed on Apr. 30, 2007, the disclosure of which is incorporated herein by reference.

### 2. BACKGROUND

This disclosure relates to systems for determining physiological characteristics.

### BRIEF DESCRIPTION OF THE DRAWINGS

FIG. 1 is a schematic illustration of an exemplary embodiment of a system for determining physiological characteristics.

FIG. 2 is a schematic illustration of an exemplary embodiment of the sensor and transmitter of the system of FIG. 1.

FIG. 3 is a schematic illustration of an exemplary embodiment of the ECG sensor of the sensor and transmitter of FIG. 2.

FIG. 4 is a schematic illustration of an exemplary embodiment of the bioimpedance sensor of the sensor and transmitter of FIG. 2.

FIG. 5 is a schematic illustration of an exemplary embodiment of the plethsymography sensor of the sensor and transmitter of FIG. 2.

FIG. 6 is a schematic illustration of an exemplary embodiment of the memory of the sensor and transmitter of FIG. 2.

FIG. 6a is a schematic illustration of an exemplary embodiment of the calculated parameters of the memory of FIG. 6.

FIG. 7 is a schematic illustration of an exemplary embodiment of the communication interface of the sensor and transmitter of FIG. 2.

FIG. 8 is a front view of the sensor and transmitter of FIG. 2.

FIG. 9 is a front view of the sensor and transmitter of FIG. 2.

FIG. 10 is a side view of the sensor and transmitter of FIG. 2.

FIG. 11 is a schematic illustration of an exemplary embodiment of the host of the system of FIG. 1.

FIG. 12 is a schematic illustration of an exemplary embodiment of the memory of the host of FIG. 11.

FIG. 13 is a schematic illustration of an exemplary embodiment of the patient records of the memory of FIG. 12.

FIGS. 14a and 14b are flow chart illustrations of an exemplary embodiment of a method for determining physiological characteristics.

FIG. 15 is a flow chart illustration of an exemplary embodiment of a method for determining blood flow.

FIG. 16 is a flow chart illustration of an exemplary embodiment of a method for determining personal norms for physiological characteristics.

FIG. 17 is a graphical illustration of exemplary experimental results in a clinical trial.

FIG. 18 is a graphical illustration of exemplary experimental results in a clinical trial.

FIG. 19 is a graphical illustration of exemplary experimental results in a clinical trial.

### DETAILED DESCRIPTION

In the drawings and description that follows, like parts are marked throughout the specification and drawings with the same reference numerals, respectively. The drawings are not necessarily to scale. Certain features of the invention may be shown exaggerated in scale or in somewhat schematic form and some details of conventional elements may not be shown in the interest of clarity and conciseness. The present invention is susceptible to embodiments of different forms. Specific embodiments are described in detail and are shown in the drawings, with the understanding that the present disclosure is to be considered an exemplification of the principles of the invention, and is not intended to limit the invention to that illustrated and described herein. It is to be fully recognized that the different teachings of the embodiments discussed below may be employed separately or in any suitable combination to produce desired results. The various characteristics mentioned above, as well as other features and characteristics described in more detail below, will be readily apparent to those skilled in the art upon reading the following detailed description of the embodiments, and by referring to the accompanying drawings.

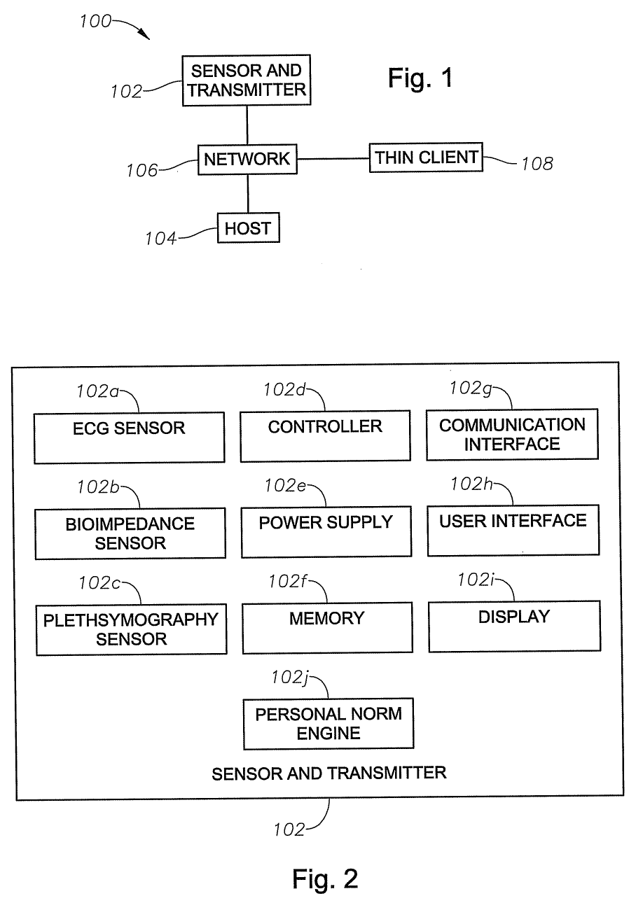

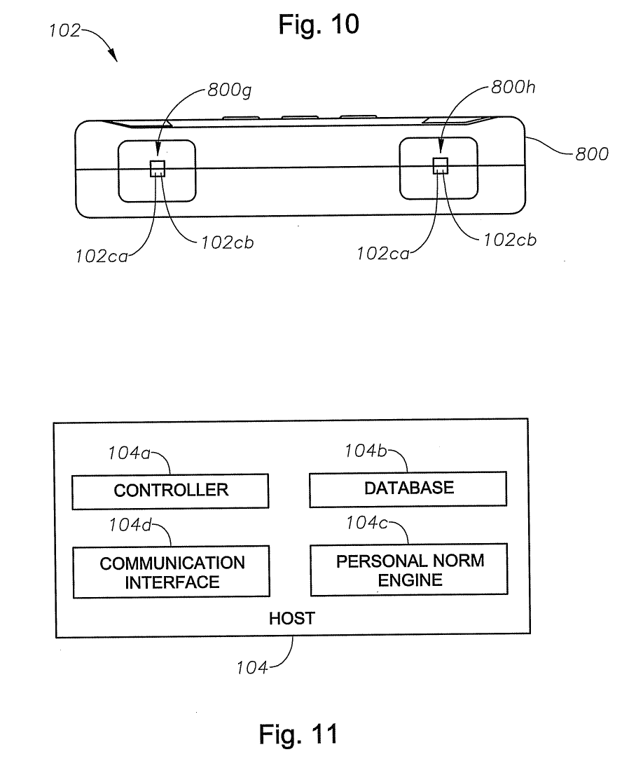

Referring initially to FIGS. 1-13, an exemplary embodiment of a system 100 for determining physiological characteristics includes one or more sensor and transmitter devices 102 that are operably coupled to a host 104 by a network 106. In an exemplary embodiment, one or more thin clients 108 are also operably coupled to the device 102 and host 104 by the network 106. In an exemplary embodiment, the network 106 in a conventional commercially available network and may, for example, include the Internet.

As illustrated in FIG. 2, an exemplary embodiment, of the device 102 includes an electrocardiogram (“ECG”) sensor 102a, a bioimpedance sensor 102b, and a plethsymography (“PLETH”) sensor 102c that are operably coupled to a controller 102d. In an exemplary embodiment, the ECG sensor 102a is adapted to obtain an ECG signal from a user of the device 102, the bioimpedance sensor 102b is adapted to obtain a bioimpedance signal from a user of the device, and the PLETH sensor 102c is adapted to obtain a PLETH signal from a user of the device.

A controller 102d is operably coupled to the ECG sensor 102a, the bioimpedance sensor 102b, and the PLETH sensor 102c for monitoring and controlling the operation of the ECG sensor, the bioimpedance sensor, and the PLETH sensor. In an exemplary embodiment, the controller 102d may include a conventional commercially available controller such as, for example, a computer processor.

A power supply 102e, a memory 102f, a communication interface 102g, a user interface 102h, a display 102i, and a personal norm engine 102j are operably coupled to the controller 102d.

In an exemplary embodiment, the power supply 102e is a conventional power supply.

In an exemplary embodiment, the memory 102f is a conventional memory device such as, for example, a flash memory device.

In an exemplary embodiment, the communication interface 102g is a conventional communication interface device adapted to permit communications between the device 102 and the network 106.

In an exemplary embodiment, the user interface 102h is a conventional user interface that is adapted to permit a user to interface with the device 102.

In an exemplary embodiment, the display 102i is a conventional display device.

In an exemplary embodiment, the personal norm engine 102j is adapted to process the ECG signals obtained by the ECG sensor 102a, the bioimpedance signal obtained by the bioimpedance sensor 102b, and/or the PLETH signal obtained by the PLETH sensor 102c to calculate one or more personal norm values that are representative of one or more normative physiological characteristics of a corresponding user of the device 102. In an exemplary embodiment, the normative physiological characteristics of a corresponding user of the device 102 include one or more of the following: a) systolic time interval; b) peak to peak variation in ECG; c) QRS length in ECG; d) pulse wave duration in PLETH; and e) bioimpedance.

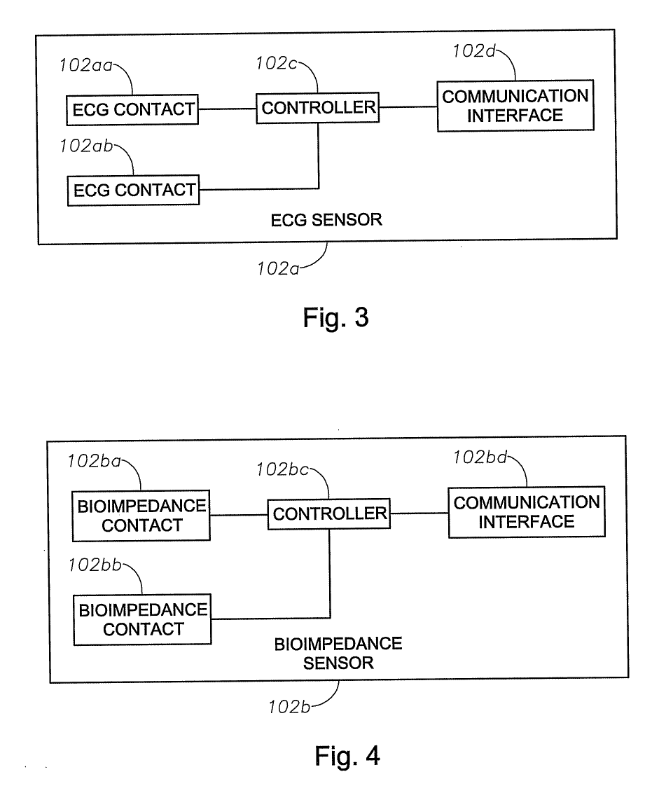

As illustrated in FIG. 3, in an exemplary embodiment, the ECG sensor 102a includes ECG contacts, 102aa and 102ab, that are operably coupled to a controller 102ac. In an exemplary embodiment, the controller 102ac is operably coupled to a communication interface 102ad for communicating with the controller 102d of the device 102. In an exemplary embodiment, the ECG contacts, 102aa and 102ab, and the controller 102ac are conventional and are adapted to obtain ECG signals from a user of the device 102 in a conventional manner.

As illustrated in FIG. 4, in an exemplary embodiment, the bioimpedance sensor 102b includes bioimpedance contacts, 102ba and 102bb, that are operably coupled to a controller 102bc. In an exemplary embodiment, the controller 102bc is operably coupled to a communication interface 102bd for communicating with the controller 102d of the device 102. In an exemplary embodiment, the bioimpedance contacts, 102ba and 102bb, and the controller 102bc are conventional and are adapted to obtain bioimpedance signals from a user of the device 102 in a conventional manner.

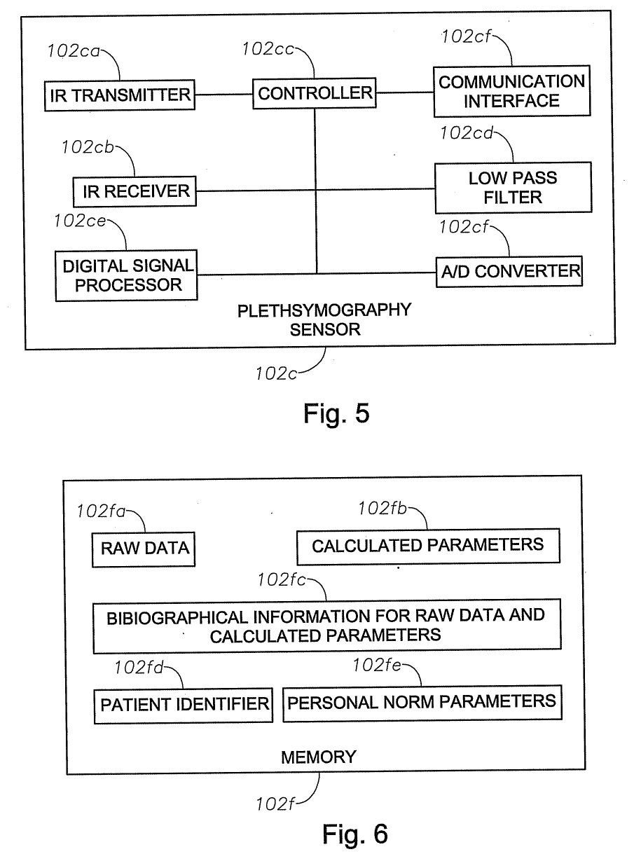

As illustrated in FIG. 5, in an exemplary embodiment, the PLETH sensor 102c includes an infrared (“IR”) transmitter 102ca, an IR receiver 102cb, and a controller 102cc operably coupled to the IR transmitter and IR receiver. A low pass filter 102cd, a digital signal processor (“DSP”) 102ce, and an A/D converter 102cf are also operably coupled to the controller 102cc. In an exemplary embodiment, the controller 102cc is further operably coupled to a communication interface 102cf for communicating with the controller 102d of the device 102. In an exemplary embodiment, the IR transmitter 102ca is adapted to transmit IR waves out of the device 102 and reflect the IR waves off of a user of the device. The reflected IR waves are then detected by the IR receiver 102cb and processed by the controller 102cc, low pass filter 102cd, DSP 102ce, and A/D converter 102cf to generate PLETH signals.

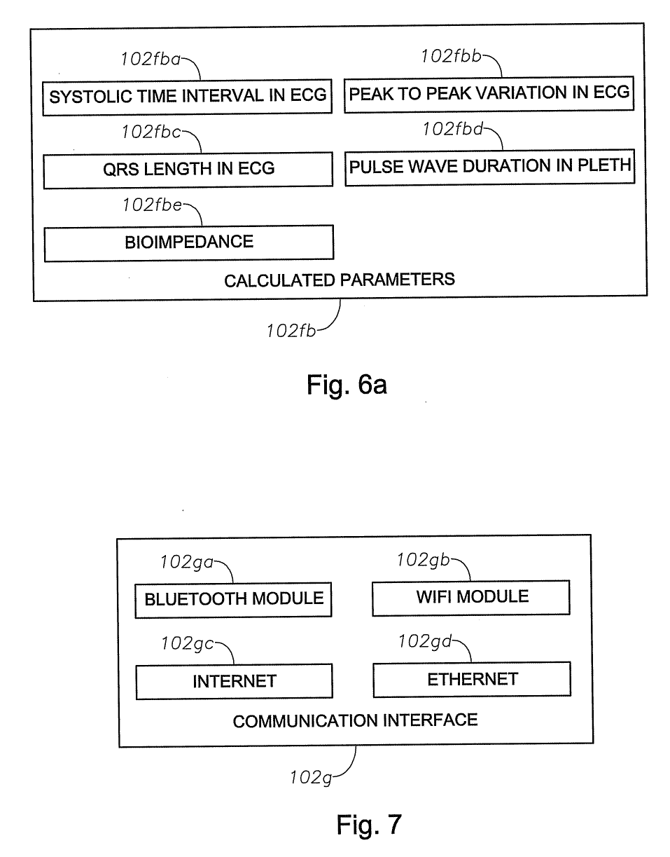

As illustrated in FIGS. 6 and 6a, in an exemplary embodiment, the memory 102f includes one or more data records representative of raw data 102fa, calculated parameters 102fb, biographical information related to the raw data and calculated parameters 102fc, patient identifier 102fd, and personal norm parameters 102fe. In an exemplary embodiment, the raw data 102fa includes data such as ECG signals, bioimpedance signals, and PLETH signals. In an exemplary embodiment, the calculated parameters 102fb include the systolic time interval 102fba; the peak to peak variation in ECG 102fbb; the QRS length in ECG 102fbc; the pulse wave duration in PLETH 102fbd; and the bioimpedance 102fbe. In an exemplary embodiment, the biographical information related to the raw data and calculated parameters 102fc include information such as the date and time of the associated raw data and/or calculated parameters. In an exemplary embodiment, the patient identifier 102fd includes a unique identification code associated with a user of the device 102. In an exemplary embodiment, the personal norm parameters 102fe include one or more normative parameters derived from the raw data and/or calculated parameters that are reflective of average parameter values for a specific user of the device 102.

As illustrated in FIG. 7, in an exemplary embodiment, the communication interface 102g of the device 102 includes a conventional Bluetooth communication module 102ga, a conventional WIFI communication module 102gb, a conventional Internet communication module 102gc, and a conventional Ethernet communication module 102gd to permit communication between the device 102 and the network 106.

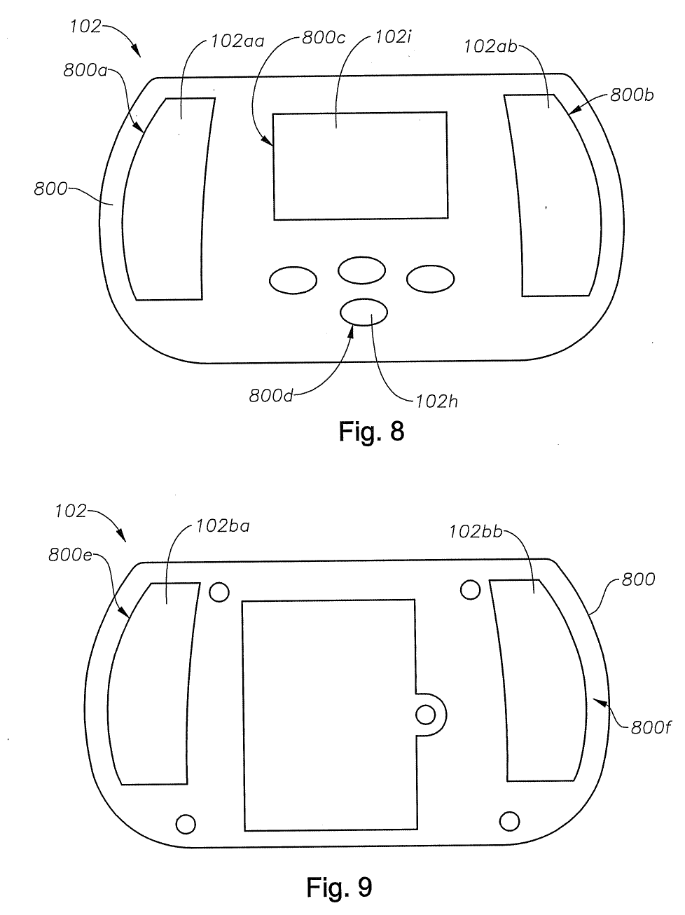

As illustrated in FIGS. 8-10, the device 102 is housed within and supported by a housing 800 that includes apertures, 800a and 800b, for the ECG contacts, 102aa and 102ab, respectively, an aperture 800c for the display 102i, one or more apertures 800d for the user interface 102h, on a front side of the housing, apertures, 800e and 800f, for the bio-impedance contacts, 102ba and 102bb, on a rear side of the housing, and apertures, 800g and 800h, that permit pairs of IR transmitters and receivers, 102ca and 102cb, positioned at each aperture, to transmit and receive IR signals.

In an exemplary embodiment, during the operation of the device 102, in order to obtain an ECG signal from a user of the device, the user grasps one of the ECG contacts, 102aa and 102ab, in each hand. In an exemplary embodiment, during the operation of the device 102, in order to obtain a bioimpedance signal from a user of the device, the user grasps one of the bioimpedance contacts, 102ba and 102bb, in each hand. In an exemplary embodiment, during the operation of the device 102, in order to obtain a PLETH signal from a user of the device, the user positions a fingertip proximate one of the apertures, 800g and 800h, that permit pairs of IR transmitters and receivers, 102ca and 102cb, positioned at each of these apertures to transmit IR signals and receive IR signals reflected by a user of the device.

As illustrated in FIG. 11, in an exemplary embodiment, the host 104 includes a controller 104a that is operably coupled to a database 104b, a personal norm engine 104c, and a communication interface 104d. In an exemplary embodiment, the controller 104a is a conventional programmable control device. In an exemplary embodiment, the database 104b includes one or more records representative of one or more physiological characteristics of one or more corresponding users of one or more device 102. In an exemplary embodiment, the personal norm engine 104c is adapted to process one or more of the records in the database 104b to generate one or more normative physiological parameters corresponding to particular users of one or more of the devices 102. In an exemplary embodiment, the communication interface 104d is a conventional communication interface that is adapted to permit communication between the host 104 and the network 106.

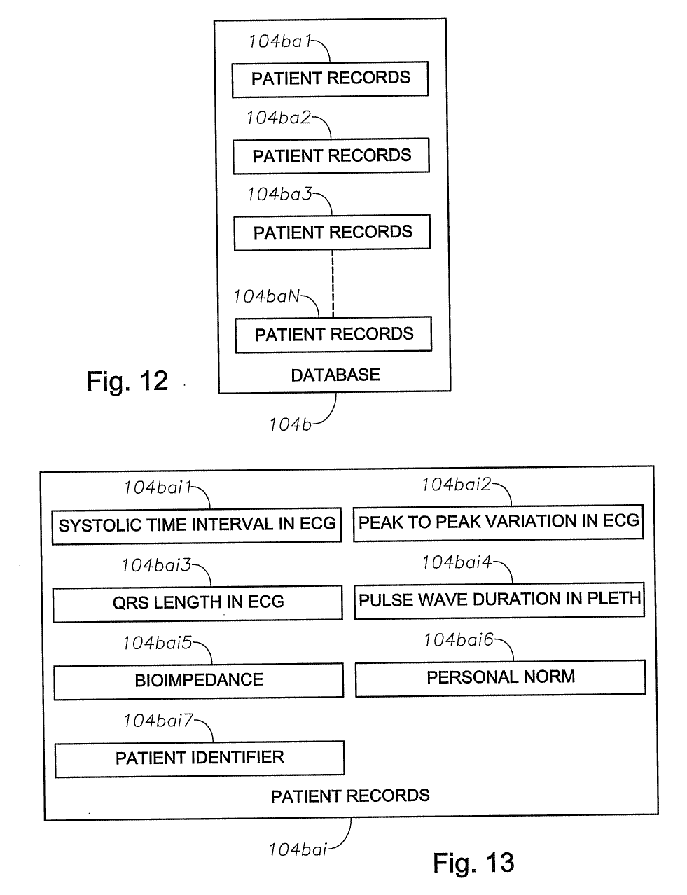

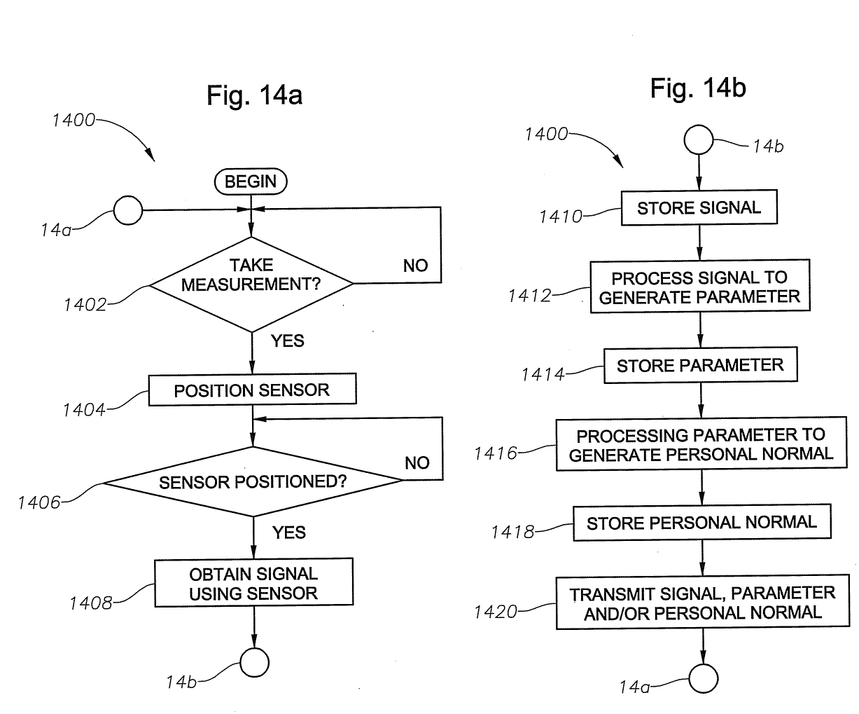

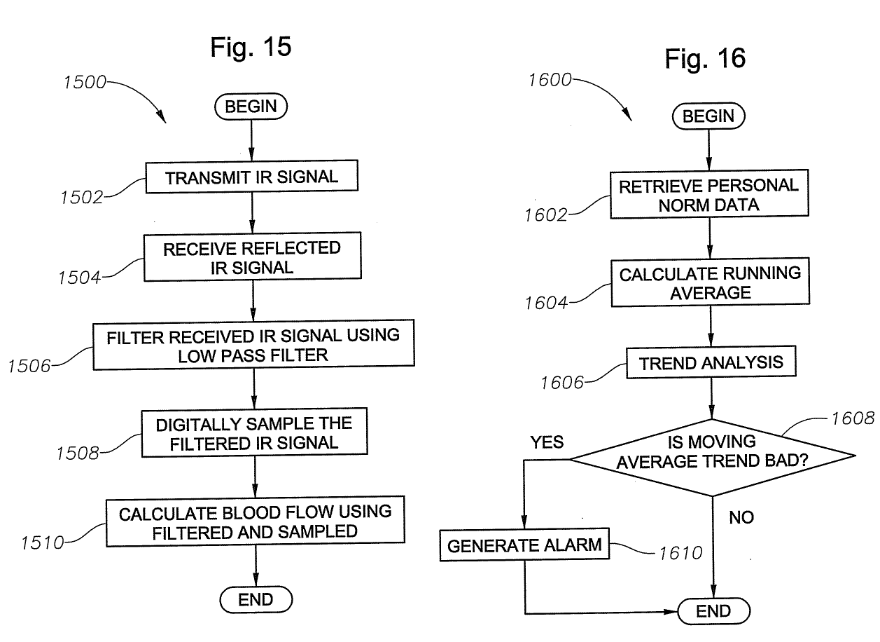

As illustrated in FIGS. 12 and 13, in an exemplary embodiment, the database 104b includes patient records 104bai, where i ranges from 1 to N. In an exemplary embodiment, the patient records 104bai include data records representative of the systolic time interval 102bai1; the peak to peak variation in ECG 102bai2; the QRS length in ECG 102bai3; the pulse wave duration in PLETH 102bai4; the bioimpedance 102bai5, one or more personal normative values 104bai6, and a unique patient identifier 104bai7. In an exemplary embodiment, the personal normative values 104bai6 associated with the unique patient identifier 104bai7 include average values of one or more of the systolic time interval 102bai1; the peak to peak variation in ECG 102bai2; the QRS length in ECG 102bai3; the pulse wave duration in PLETH 102bai4; the bioimpedance 102bai5 which may, for example, include an overall average, a running average, and a trend line associated with one or more running averages.

In an exemplary embodiment, during the operation of the system 100, the system 100 implements a method 1400 of measuring one or more physiological characteristics in which, in 1402, a user of the device 102 may elect to take a physiological measurement by operating the user interface 102h of the device. If the user of the device 102 elects to take a measurement, then the user may then position the device to take the measurement in 1404.

In an exemplary embodiment, in 1404, during the operation of the device 102, in order to obtain an ECG signal from a user of the device, the user grasps one of the ECG contacts, 102aa and 102ab, in each hand. In an exemplary embodiment, in 1404, during the operation of the device 102, in order to obtain a bioimpedance signal from a user of the device, the user grasps one of the bioimpedance contacts, 102ba and 102bb, in each hand. In an exemplary embodiment, in 1404, during the operation of the device 102, in order to obtain a PLETH signal from a user of the device, the user positions a fingertip proximate one of the apertures, 800g and 800h, that permit pairs of IR transmitters and receivers, 102ca and 102cb, positioned at each of these apertures to transmit IR signals and receive IR signals reflected by a user of the device.

If the user has positioned the device in 1406 in order to take a measurement, then, in 1408, the device 1408 obtains the selected physiological signal in 1408. In an exemplary embodiment, the selected physiological signal may include an ECG signal, a bioimpedance signal, or a PLETH signal. In an exemplary embodiment, in 1408, a user may of the device 102 may initiate the obtaining of the selected physiological signal by, for example, depressing a push button provided on the user interface 102h.

In an exemplary embodiment, the physiological signal obtained in 1408 is then stored in 1408 in the memory 102f in one or more of the raw data records 102fa in the memory of the device 102.

In an exemplary embodiment, the signal stored in the memory 102f of the device is then processed to generate a parameter representative of a physiological characteristic in 1412. In an exemplary embodiment, the parameter generated in 1412 may include the systolic time interval, the peak to peak variation in ECG, the QRS length in ECG, the pulse wave duration in PLETH, and/or the bioimpedance.

In an exemplary embodiment, the parameter calculated in 1412 is then stored in 1414 in the memory 102f in one or more of the data records 102fb in the memory of the device 102.

In an exemplary embodiment, one or more of the parameters generated and stored in 1412 and 1414 are then processed to generate one or more personal normative values for the user of the device 102 in 1416. In an exemplary embodiment, the personal normative values may include average values for the parameters that may, for example, include overall average values, running average values, trends in overall averages, trends in running averages, and/or deviations in individual or trend values from other average an/or trend values.

In an exemplary embodiment, the personal normative values generated in 1416 are then stored in the memory 102f of the device 102 in one or more of the personal normative value data records 102fe in 1418.

In an exemplary embodiment, in 1420, one or more of the data records representative of raw data 102fa, calculated parameters 102fb, biographical information related to the raw data and calculated parameters 102fc, patient identifier 102fd, and personal norm parameters 102fe may be transmitted to the host 104 by the device 102.

In an exemplary embodiment, during operation of the system 100, the system implements a method 1500 of calculating a parameter representative of blood flow within a user of one of the devices 102 by, in 1502, transmitting an IR signal from the IR transmitter 102ca of the device onto the skin surface of the user of the device. In 1504, the IR signal reflected by the skin surface of the user of the device 102 is received by the IR receiver 102cb of the device.

In an exemplary embodiment, the IR signal received in 1504 is then filtered in 1506 using the low pass filter 102cd of the device 102 in 1506.

In an exemplary embodiment, the low pass filtered IR signal is then digitally sampled and processed in 1508 by the DSP 102ce and the A/D converter 102cf of the device 102 in 1508. In an exemplary embodiment, in 1508, the low pass filtered IR signals is processed by the A/D converter 102cf prior to being processed by the DSP 102ce of the device 102.

In an exemplary embodiment, the digitally sampled IR signal is then processed in a conventional manner in 1510 to determine the parameter representative of blood flow within the user of the device 102 in 1510.

In an exemplary embodiment, as illustrated in FIG. 16, during the operation of the system 100, the system implements a method 1600 of determining if a personal normative value is indicative of a need for further medical evaluation in which, in 1602, normative data associated with a particular user is retrieved. In an exemplary embodiment, in 1602, the personal normative data associated with a particular user may be retrieved from the memory 102f of one or more of the devices 102 and/or the database 104b of the host 104. In an exemplary embodiment, the personal normative data may include personal normative data associated with one or more of the following: systolic time interval, the peak to peak variation in ECG, the QRS length in ECG, the pulse wave duration in PLETH, and/or the bioimpedance.

In an exemplary embodiment, in 1604, the running average of one or more of the retrieved personal normative data is calculated.

In an exemplary embodiment, in 1606, a trend analysis of the running average calculated in 1604 is provided.

In an exemplary embodiment, in 1608, if the trend of the moving average indicates a need for further medical evaluation, then an alarm is generated in 1610 which may, for example, include a visual alarm, an audible alarm, or an email alert.

In several exemplary embodiment, the method 1600 may be implemented in whole or in part by the device 102, the host 104 or the thin client 108.

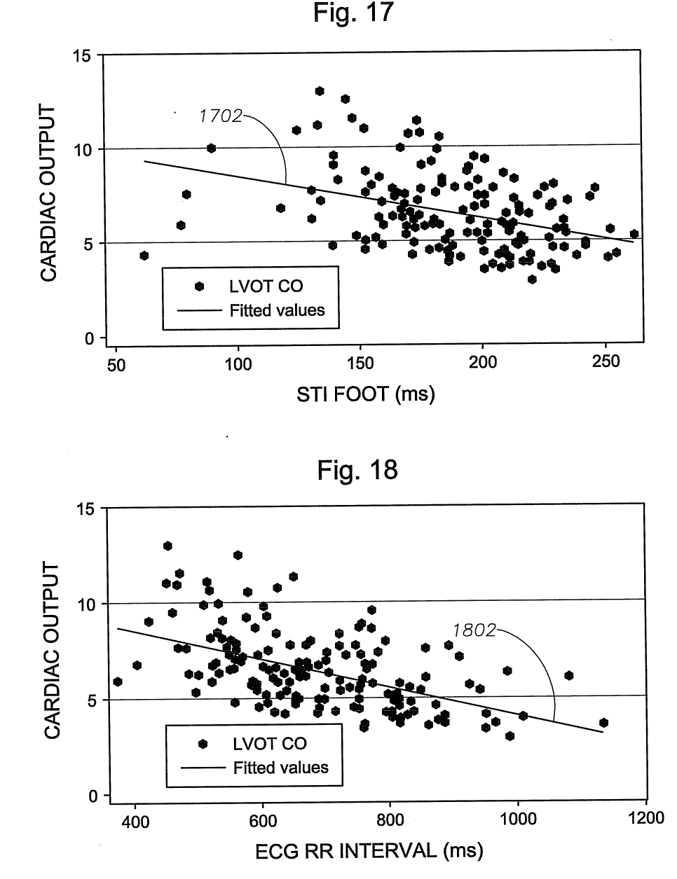

In an exemplary clinical trial, as illustrated in FIG. 17, patient data was obtained from a number of patients in the clinical trial that indicated a predictive relationship 1702 between systolic time interval in ECG and cardiac output. Thus, a measurement of the systolic time interval in ECG using the system 100 of the present exemplary embodiments will provide an effective non-invasive proxy of also determining the cardiac output of a user of the system. This was an unexpected result of the clinical trial.

In an exemplary clinical trial, as illustrated in FIG. 18, patient data was obtained from a number of patients in the clinical trial that indicated a predictive relationship 1802 between peak to peak variation in ECG and cardiac output. Thus, a measurement of the peak to peak variation in ECG using the system 100 of the present exemplary embodiments will provide an effective non-invasive proxy of also determining the cardiac output of a user of the system. This was an unexpected result of the clinical trial.

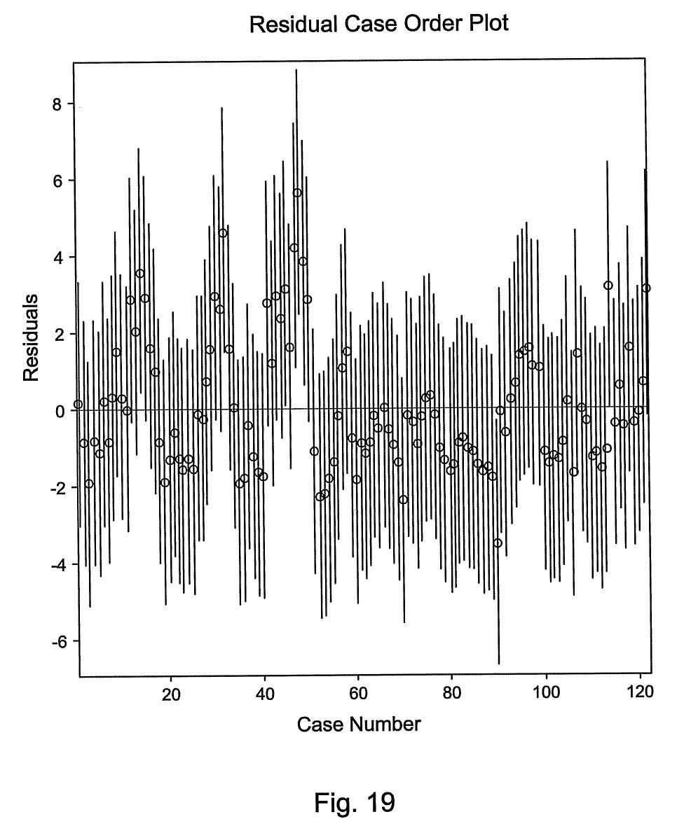

In an exemplary clinical trial, as illustrated in FIG. 19, the patient data of the clinical trials illustrated and described above with reference to FIGS. 17 and 18, was further processed by performing a multiple linear regression of the combined predictive powers of the predictive relationships, 1702 and 1802. As illustrated in FIG. 19, the residuals of the multiple linear regression performed indicates a strong correlation between the multiple linear regression of the combined predictive powers of the predicative relationships, 1702 and 1802, and the cardiac output of the patients. This was an unexpected result of the clinical trial.

In an exemplary embodiment, during the operation of the system 100, the systolic time interval is generated in a conventional manner by processing the ECG and PLETH signals obtained by the device 102.

In an exemplary embodiment, the processing of the digitally sampled IR signal to determine the parameter representative of blood flow within the user of the device in 1510 is provided using the Beer-Lambert Law.

In an exemplary embodiment, in 1604, the calculation of the running average of one or more of the retrieved personal normative data includes an analysis of diurnal variation of the retrieved personal normative data.

In an exemplary embodiment, in 1606, a trend analysis of the running average calculated in 1604 is provided.

In an exemplary embodiment, in 1608, if the trend of the moving average indicates a need for further medical evaluation, including, for example, information gap analysis and/or other mathematical analysis, then an alarm is generated in 1610 which may, for example, include a visual alarm, an audible alarm, or an email alert.

In an exemplary embodiment, if the value of any of the parameters generated by the system 100 indicate a need for further medical evaluation, including, for example, information gap analysis and/or other mathematical analysis, then an alarm may be generated which may, for example, include a visual alarm, an audible alarm, or an email alert.

In an exemplary embodiment, the parameters provided by the system 100 may also be used as predictors of cardiac decompensation which is typically the chief cause of mortality for patients with heart failure. In addition, the parameters provided by the system 100 may also be used as predictors of autonomic control, vascular compliance, fluid retention, and myocardial performance.

In several exemplary embodiments, the elements and operations of the exemplary embodiments may be provided by one or more devices 102, hosts 104, or distributed between and among the devices and hosts.

It is understood that variations may be made in the above without departing from the scope of the invention. For example, as one measure of autonomic control, the device 102 could be used as part of a reflex detection system such as, for example, a lie detector. In addition, the system 100 could be used to help treat medical disorders by using the bioimpedance parameter as a proxy for fluid retention which may facilitate the treatment of edema. Furthermore, the teachings of the present exemplary embodiments may be extended to the determination of physiological characteristics for human and/or animal subjects. Further, spatial references are for the purpose of illustration only and do not limit the specific orientation or location of the structure described above. While specific embodiments have been shown and described, modifications can be made by one skilled in the art without departing from the spirit or teaching of this invention. The embodiments as described are exemplary only and are not limiting. Many variations and modifications are possible and are within the scope of the invention. Accordingly, the scope of protection is not limited to the embodiments described, but is only limited by the claims that follow, the scope of which shall include all equivalents of the subject matter of the claims.

1. A system for determining one or more physiological characteristics, comprising:
a communication network;
a remote sensor operably coupled to the network adapted to sense and record one or more physiological characteristics; and
a host computer operably coupled to the network for receiving one or more of the physiological characteristics sensed and recorded by the remote sensor;
wherein at least one of the remote sensor and host computer are adapted to process the sensed and recorded physiological characteristics to determine one or more corresponding normative physiological parameters for a corresponding user of the remote sensor.

2. The system of claim 1, wherein the normative physiological parameter comprises a proxy for cardiac output for a corresponding user of the remote sensor.

3. The system of claim 1, wherein the remote sensor comprises:
an ECG sensor;
a bioimpedance sensor; and
a plethsymography sensor.

4. The system of claim 1, wherein the remote sensor comprises:
a memory for storing one or more physiological characteristics;
a communication interface for communicating with the network; and
a personal norm engine for processing the sensed and recorded physiological characteristics to determine one or more corresponding normative physiological parameters for a corresponding user of the remote sensor.

5. The system of claim 4, wherein the memory comprises:
one or more data records representative of sensed physiological characteristics;
one or more data records representative of physiological parameters calculated from the sensed physiological characteristics; and
one or more normative physiological parameters for a corresponding user of the remote sensor calculated from the physiological parameters.

6. The system of claim 5, wherein the physiological parameters comprises:
a systolic time interval in an ECG signal and a plethsymography signal;
a peak to peak variation in an ECG signal;
a QRS length in an ECG signal;
a pulse wave duration in a plethsymography signal; and
a bioimpedance value.

7. The system of claim 5, wherein the memory comprises:
one or more data records representative of biographical information associated with the sensed physiological characteristics; and
one or more data records representative of biographical information associated with physiological parameters calculated from the sensed physiological characteristics.

8. The system of claim 5, wherein the memory comprises:
one or more data records representative of patient identifiers associated with the sensed physiological characteristics; and
one or more data records representative of patient identifiers associated with physiological parameters calculated from the sensed physiological characteristics.

9. The system of claim 1, wherein the remote sensor comprises:
a plethsymography sensor comprising:
a controller;
an IR transmitter operably coupled to the controller for transmitting IR signals;
an IR receiver operably coupled to the controller for receiving IR signals; and
a low pass filter operably coupled to the IR receiver for filtering the received IR signals.

10. The system of claim 1, wherein the host computer comprises:
a memory for storing one or more physiological characteristics;
a communication interface for communicating with the network; and
a personal norm engine for processing the sensed and recorded physiological characteristics to determine one or more corresponding normative physiological parameters for a corresponding user of the remote sensor.

11. The system of claim 10, wherein the memory comprises:
one or more data records representative of sensed physiological characteristics;
one or more data records representative of physiological parameters calculated from the sensed physiological characteristics; and
one or more normative physiological parameters for a corresponding user of the remote sensor calculated from the physiological parameters.

12. The system of claim 11, wherein the physiological parameters comprises:
a systolic time interval in an ECG signal;
a peak to peak variation in an ECG signal;
a QRS length in an ECG signal;
a pulse wave duration in a plethsymography signal; and
a bioimpedance value.

13. The system of claim 11, wherein the memory comprises:
one or more data records representative of biographical information associated with the sensed physiological characteristics; and
one or more data records representative of biographical information associated with physiological parameters calculated from the sensed physiological characteristics.

14. The system of claim 11, wherein the memory comprises:
one or more data records representative of patient identifiers associated with the sensed physiological characteristics; and
one or more data records representative of patient identifiers associated with physiological parameters calculated from the sensed physiological characteristics.

15. The system of claim 1, further comprising one or more thin clients operably coupled to the network for remotely accessing the host computer for accessing one or more of the physiological characteristics and normative physiological parameters for a corresponding user of the remote sensor.

16. The system of claim 15, wherein the normative physiological parameter comprises a cardiac output for a corresponding user of the remote sensor.

17. An apparatus for determining one or more physiological characteristics, comprising:
a sensor adapted to sense and record one or more physiological characteristics;
wherein the sensor is adapted to process the sensed and recorded physiological characteristics to determine one or more corresponding normative physiological parameters for a corresponding user of the remote sensor.

18. The apparatus of claim 17, wherein the normative physiological parameter comprises a proxy for a cardiac output for a corresponding user of the sensor.

19. The apparatus of claim 17, wherein the sensor comprises:
an ECG sensor;
a bioimpedance sensor; and
a plethsymography sensor.

20. The apparatus of claim 17, wherein the sensor comprises:
a memory for storing one or more physiological characteristics;
a communication interface for communicating with the network; and
a personal norm engine for processing the sensed and recorded physiological characteristics to determine one or more corresponding normative physiological parameters for a corresponding user of the sensor.

21. The apparatus of claim 20, wherein the memory comprises:
one or more data records representative of sensed physiological characteristics;
one or more data records representative of physiological parameters calculated from the sensed physiological characteristics; and
one or more normative physiological parameters for a corresponding user of the sensor calculated from the physiological parameters.

22. The apparatus of claim 21, wherein the physiological parameters comprises:
a systolic time interval in an ECG signal and a plethsymography signal;
a peak to peak variation in an ECG signal;
a QRS length in an ECG signal;
a pulse wave duration in a plethsymography signal; and
a bioimpedance value.

23. The apparatus of claim 21, wherein the memory comprises:
one or more data records representative of biographical information associated with the sensed physiological characteristics; and
one or more data records representative of biographical information associated with physiological parameters calculated from the sensed physiological characteristics.

24. The apparatus of claim 21, wherein the memory comprises:
one or more data records representative of patient identifiers associated with the sensed physiological characteristics; and
one or more data records representative of patient identifiers associated with physiological parameters calculated from the sensed physiological characteristics.

25. The apparatus of claim 17, wherein the sensor comprises:
a plethsymography sensor comprising:
a controller;
an IR transmitter operably coupled to the controller for transmitting IR signals;
an IR receiver operably coupled to the controller for receiving IR signals; and
a low pass filter operably coupled to the IR receiver for filtering the received IR signals.

26. A method of determining one or more physiological characteristics, comprising:
sensing and recording one or more physiological characteristics at a remote location;
transmitting the remotely sensed and recorded physiological characteristics to a host computer; and
processing the sensed and recorded physiological characteristics to determine one or more corresponding normative physiological parameters for a corresponding user.

27. The method of claim 26, wherein the normative physiological parameters comprise a proxy for a cardiac output for a corresponding user of the remote sensor.

28. The method of claim 26, wherein the physiological characteristics comprise:
an ECG signal;
a bioimpedance signal; and
a plethsymography signal.

29. The method of claim 26, further comprising:
remotely storing one or more physiological characteristics; and
remotely processing the sensed and recorded physiological characteristics to determine one or more corresponding normative physiological parameters for a corresponding user of the remote sensor.

30. The method of claim 29, further comprising:
remotely storing one or more data records representative of sensed physiological characteristics;
remotely storing one or more data records representative of physiological parameters calculated from the sensed physiological characteristics; and
remotely storing one or more normative physiological parameters for a corresponding user of the remote sensor calculated from the physiological parameters.

31. The method of claim 30, wherein the physiological parameters comprise:
a systolic time interval in an ECG signal and a plethsymography signal;
a peak to peak variation in an ECG signal;
a QRS length in an ECG signal;
a pulse wave duration in a plethsymography signal; and
a bioimpedance value.

32. The method of claim 30, further comprising:
remotely storing one or more data records representative of biographical information associated with the sensed physiological characteristics; and
remotely storing one or more data records representative of biographical information associated with physiological parameters calculated from the sensed physiological characteristics.

33. The method of claim 30, further comprising:
remotely storing one or more data records representative of patient identifiers associated with the sensed physiological characteristics; and
remotely storing one or more data records representative of patient identifiers associated with physiological parameters calculated from the sensed physiological characteristics.

34. The method of claim 26, further comprising:
transmitting IR signals onto a user;
receiving IR signals reflected from the user; and
filtering the received IR signals using a low pass filter.

35. The method of claim 26, further comprising:
at the host computer, storing one or more physiological characteristics transmitted to the host;
at the host computer, processing the physiological characteristics to determine one or more corresponding normative physiological parameters for a corresponding remote user.

36. The method of claim 35, further comprising:
at the host computer, storing one or more data records representative of sensed physiological characteristics;
at the host computer, storing one or more data records representative of physiological parameters calculated from the sensed physiological characteristics; and
at the host computer, storing one or more normative physiological parameters for a corresponding user calculated from the physiological parameters.

37. The method of claim 36, wherein the physiological parameters comprise:
a systolic time interval in an ECG signal;
a peak to peak variation in an ECG signal;
a QRS length in an ECG signal;
a pulse wave duration in a plethsymography signal; and
a bioimpedance value.

38. The method of claim 36, further comprising:
at the host computer, storing one or more data records representative of biographical information associated with the sensed physiological characteristics; and
at the host computer, storing one or more data records representative of biographical information associated with physiological parameters calculated from the sensed physiological characteristics.

39. The method of claim 36, further comprising:
at the host computer, storing one or more data records representative of patient identifiers associated with the sensed physiological characteristics; and
at the host computer, storing one or more data records representative of patient identifiers associated with physiological parameters calculated from the sensed physiological characteristics.

40. The method of claim 36, further comprising permitting one or more thin clients to remotely access the host computer for accessing one or more of the physiological characteristics and normative physiological parameters for a corresponding user of the remote sensor.

41. The method of claim 14, wherein the normative physiological parameter comprises a cardiac output for a corresponding user.
